(19) United States
(12) Patent Application Publication
(10) Pub. No.: US 2011/0066136 A1
(43) Pub. Date: Mar. 17, 2011

(54) USE OF PUMP TO DELIVER A SOLUTION TO REDUCE SNORING

(75) Inventor: Rene Arnfeldt Moller, Holte (DK)

(73) Assignee: TANNER MEDICO A/S, Horsholm (DK)

(21) Appl. No.: 12/879,220

(22) Filed: Sep. 10, 2010

(30) Foreign Application Priority Data
Sep. 11, 2009 (CN) 200910211634.0

(51) Int. Cl. A61M 31/00; A61M 1/00

(52) U.S. Cl. 604/514; 604/151

(57) ABSTRACT

A method of delivering a fluid to a pharynx area through a nasal cavity includes bringing a pump to a nasal passage of a nose at an angle of approximately −90 to 130 degrees relative a horizontal ground plane, and pumping a jet of the fluid through the nasal passage.

### CROSS-REFERENCE TO RELATED APPLICATIONS

The present application claims priority to and the benefit of Chinese Application No. 200910211634.0, filed on 11 Sep. 2009, the disclosure of which is incorporated herein by reference in its entirety.

### FIELD

The present disclosure generally relates to a dispensing a fluid, and in particular to a dispensing an anti-snoring solution in liquid form.

### BACKGROUND

Snoring is an irritating condition where air movement during sleeping is obstructed. Snoring can be rather annoying and unpleasant. It can also cause sleep deprivation to both the person snoring and the persons around him. It is therefore beneficial to a person to reduce his snoring.

Most methods for reducing snoring revolve around clearing the blockage in the breathing passage. This is the reason snorers are advised to lose weight (to stop fat from pressing on the throat), stop smoking (smoking weakens and clogs the throat) and sleep on their side (to prevent the tongue from blocking the throat). A number of other treatment options are also available, ranging from over-the-counter aids such as nasal strips or nose clips, lubricating sprays, and “anti-snore” clothing and pillows.

These methods are not always efficient and more severe methods are often needed such as surgery, continuous airway pressure machine and antidepressant, which all affect a person's life to a great extent having to undergo surgery, always having a machine at hand or risking becoming addicted to an antidepressant drug.

Referring to FIG. 1, one common area where the air is obstructed is the pharynx 102. Various medicines, drugs and traditional healing extracts are dispensed through the use of nasal or oral spray pumps. One particular example is nasal spray, which is most often distributed via a nozzle spray pump by inserting the nozzle into a person's nose and spraying in the nasal spray by pumping.

The pharynx 102 (or pharynxes) is an area where obstructions occur easily as it is relatively narrow. It is thus highly beneficial to focus on this area to prevent the obstructions and thereby reduce the snoring. It is common to reduce a swelling by administering a solution to it, which solution reduces the swelling and thereby reduces the obstruction. However, due to the position of the pharynx 102 and the anatomy of the head 101 of a person, the pharynx 102 can be difficult to reach.

Access through the oral cavity 103 is usually not comfortable for the person as it induces a gag reflex in a person. It is also difficult to apply any solution to the pharynx 102 as the oral cavity 103 leads to the esophagus and to the lungs as well thus risking that any solution administered reaches the stomach instead of the pharynx 102 as intended. If the solution reaches the stomach it might be without effect and if the solution reaches the lungs it might be highly unpleasant to the person.

Spraying a solution through the nasal cavity 105 is difficult as the spray causes most of the solution or to hit the walls of the nose 104 and the nasal cavity 105. Furthermore the shape of the path up the nasal cavity 105 requires the solution to travel first up and then down along a specific path to be able to reach the pharynx 102 and it is difficult for a person to aim the spray pump 107 correctly to make sure that the sprayed solution does not hit the walls of the nose 104 or the nasal cavity 105.

By making the inventive and insightful realization that by titling back the head 101 of a person, a clearer path from the nose 104 through the nasal cavity 105 to the pharynx 102 is achieved. However, the head 101 of a person and therefore also the nose 104 is now at an angle that is close to vertical making it even more difficult to administer a fluid using conventional means. It would be advantageous to overcome the prejudice of traditional spray methods and other solutions are difficult to administer at an angle and using a pump that is able to repeatedly deliver fluid even when at an angle to deliver a fluid to the pharynx 102 of a person.

A manner of non-intrusively reducing snoring would thus be useful in modern day society.

Accordingly, it would be desirable to provide a system that addresses at least some of the problems identified above.

### SUMMARY

As described herein, the exemplary embodiments overcome one or more of the above or other disadvantages known in the art.

One aspect of the exemplary embodiments relates to a method. In one embodiment the method includes delivering a fluid to a pharynx area through a nasal cavity by bringing a pump to a nasal passage of a nose at an angle of approximately −90 to 130 degrees relative a horizontal ground plane, and pumping a jet of the fluid through the nasal passage.

Another aspect of the disclosed embodiments relates to a use of a pump to administer a liquid solution to a pharynx area through a nose and a nasal cavity. In one embodiment the use includes a pump to provide a jet of a liquid solution from the pump, wherein the pump is configured to provide a jet stream of the liquid solution into the nasal cavity while the pump is positioned at an angle of approximately −10 to 90 degrees relative a substantially horizontal ground plane.

In a further aspect, the disclosed embodiments relate to a nasal fluid delivery system. In one embodiment, the nasal fluid delivery system includes a pump for delivering a liquid solution through a nose and nasal cavity to a pharynx area. The pump includes a nozzle configured to generate a jet stream of the liquid solution. The pump is configured to deliver the jet stream of the liquid solution while the pump is positioned at an angle relative to a substantially horizontal ground plane.

These and other aspects and advantages of the exemplary embodiments will become apparent from the following detailed description considered in conjunction with the accompanying drawings. It is to be understood, however, that the drawings are designed solely for purposes of illustration and not as a definition of the limits of the invention, for which reference should be made to the appended claims. Moreover, the drawings are not necessarily drawn to scale and unless otherwise indicated, they are merely intended to conceptually illustrate the structures and procedures described herein. In addition, any suitable size, shape or type of elements or materials could be used.

### BRIEF DESCRIPTION OF THE DRAWINGS

In the drawings:

FIG. 1 is a side view of a person using a pump according to an embodiment;

FIG. 2 is a flowchart of an exemplary method according to an embodiment;

FIG. 3 is a cut out side view of an apparatus according to an embodiment; and

FIG. 4 is a series of views of a person using a pump according to an embodiment.

### DETAILED DESCRIPTION OF THE EXEMPLARY EMBODIMENTS OF THE DISCLOSURE

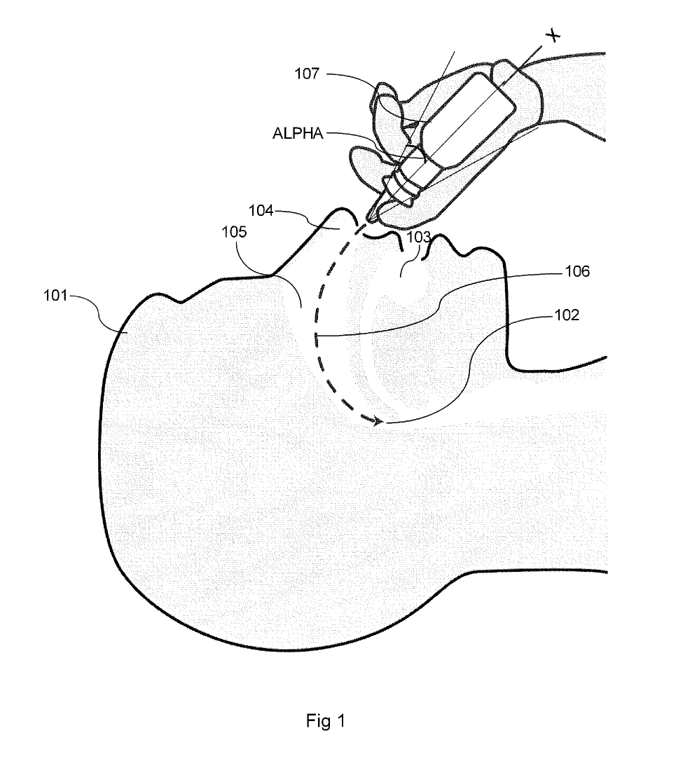

Referring to FIG. 1 an apparatus and a method of dispensing a fluid, such as an anti-snoring solution to the pharynx 102 in an efficient and effective manner will be described. The aspects of the disclosed embodiments provide for the dispensing of the fluid in the form of a jet stream even while the person's head 101 is titled back to a position that could be considered substantially or close to a vertical position with respect to the ground.

FIG. 1 illustrates a person's head 101 at rest. In this example it will be assumed that the person is lying down, but the person may also be in another position with the head 101 leaned or tilted back.

The pharynx 102 (or pharynxes) is an area where obstructions occur easily as it is relatively narrow. It is thus highly beneficial to focus on this area to prevent the obstructions and thereby reduce the snoring. It is common to reduce a swelling by administering a solution to it which solution reduces the swelling and thereby reduces the obstruction. However, due to the position of the pharynx 102 in the head 101 of a person, the pharynx 102 can be difficult to reach with traditional forms of fluid dispensers.

Access through the oral cavity 103 is usually not comfortable for the person as it induces a gag reflex in a person. It is also difficult to apply any solution to the pharynx as the oral cavity 103 leads to the esophagus and to the lungs, and there is the risk that any solution administered reaches the stomach instead of the pharynx as intended. If the solution reaches the stomach it might be without effect and if the solution reaches the lungs it might be highly unpleasant to the person.

Spraying a solution through the nasal cavity 105 is difficult as the spray generally causes most of the solution or to hit the walls of the nose 104 and the nasal cavity 105. Furthermore the shape up the passage of the nasal cavity 105 requires the solution to be able to travel first up and then down along a specific path to be able to reach the pharynx 102. It is also difficult for a person to aim the spray pump correctly to make sure that the sprayed solution does not hit the walls of the nose 104 or the nasal cavity 105.

By making the inventive and insightful realization that by titling back the head 101 of a person a clearer path from the nose 104 through the nasal cavity 105 to the pharynx 102 is achieved. However, the head 101 of a person and therefore also the nose 104 is now at an angle that is close to vertical relative to the ground, making it even more difficult to administer a fluid using conventional means. By further realizing and overcoming the prejudice that solutions are difficult to administer at an angle and using a pump that is able to repeatedly deliver fluid even when at an angle and to make the thinking leap of delivering a fluid in a jet instead of the traditional spray, the aspects of the disclosed embodiments provide a method of delivering a fluid to the pharynx 102 of a person is achieved.

As is shown in FIG. 1, the head 101 of a person is tilted backwards and a pump 107 is used to dispense a jet of fluid 106 which travels essentially along a path trough the nose 104 and nasal cavity 105 to reach the pharynx 102. The method of providing the solution or fluid to the pharynx 102 is easy to use and non-obtrusive. For example, the risk of gagging is almost non-existent as the area in the back of the oral cavity is not tampered with and even if the person was to swallow some of the fluid the fluid would still have passed the pharynx 102 and thus have some effect for reducing any obstruction.

As the solution enters the pharynx area 102 it comes into contact with the mucus membranes which can then be caused to contract thereby reducing any obstruction caused by the mucus membranes. To ensure that the solution reaches the pharynx area 102 the fluid should be repeatedly dispensed by pumping 2 to 8 times. By pumping 4 to 6 times enough fluid will have reached the pharynx area 102 using a pump according to the method described herein. As a person goes to sleep every night it is important that the delivery of the solution or fluid is easy to execute and does not bring about any unpleasant side-effects or other unpleasantries such as the gag-reflex discussed above.

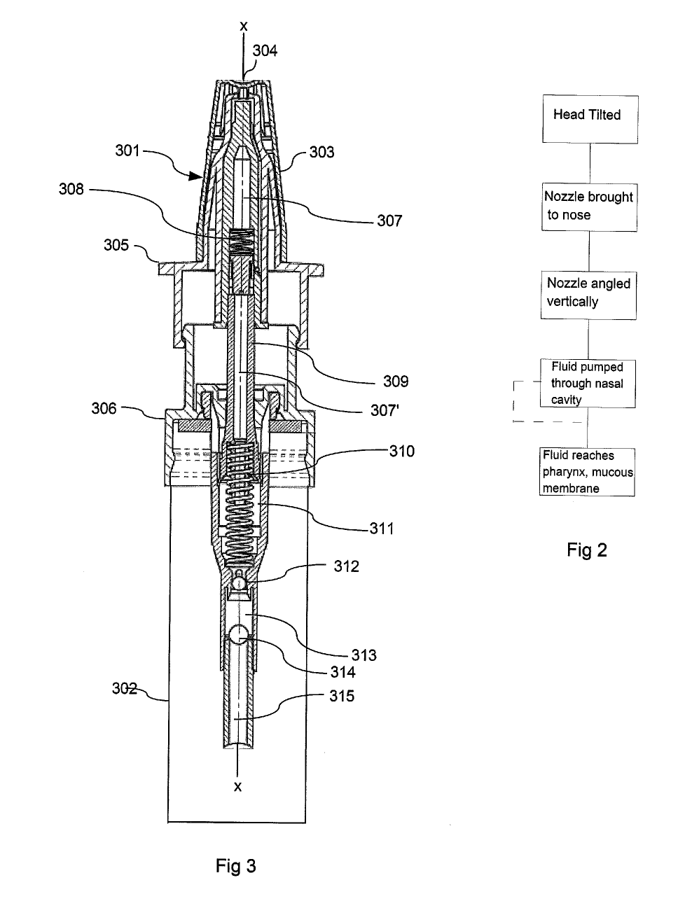

FIG. 2 shows a flowchart of a general method and use of a pump to deliver a fluid or solution 106 to the pharynx area 102 of a person which will now be described with simultaneous reference to FIGS. 1 and 3.

First the head 101 is tilted 210. Secondly, a nozzle of a pump 107 carrying the fluid is brought 220 to the nose 104 so that the nozzle rests at an angle being vertical or even pointing downwards 230, relative to the ground.

As shown in FIG. 1, a first angle marked as ALPHA indicates the angle of the pump 107 relative to the person 101. The angle ALPHA needed to distribute the fluid to the pharynx 102 in accordance with the aspects of the disclosed embodiments varies from individual to individual, but is in the order of −30 to 30 degrees as seen from a center line X-X through the pump 107.

The first angle is indicated in FIG. 1 as ALHPA and denotes the angle at which the pump 107 is being held or positioned. Being substantially vertical means that ALPHA is close to 0 (zero). Different values of the ranges of the angle ALPHA can include for example, approximately −60 to 60 degrees; −50 to 50 degrees; −40 to 40 degrees; −30 to 30 degrees; −20 to 20 degrees; −10 to 10 degrees and −5 to 5 degrees.

The fluid is pumped 240 in a jet 106 through the nasal cavity 105 reaching 250 the pharynx area 102 and thereby the mucous membrane. In one embodiment, the pumping 240 is repeated 2 to 8 times (preferably 4 to 6 times) to ensure that enough fluid has reached the pharynx area 102. The repetition of the pumping 240 is indicated in FIG. 2 with a dashed line.

The pump 107 is of a kind capable of delivering fluid repeatedly while being held at an angle being close to vertical or even tilted upside down or slightly upside down. The nozzle 304 of the pump 107 is arranged to advantageously deliver or dispense the fluid in the form of a jet or jet stream, rather than a spray of fluid.

FIG. 3 illustrates one embodiment of a pump device 301 for a fluid dispenser 107 incorporating aspects of the disclosed embodiments. The pump 301 is to be attached to a container 302. The container 302 is a vessel for carrying the fluid to be dispensed.

The pump 301 is able to repeatedly deliver the fluid even when tilted at angles close to vertical and while being upside-down. Examples of such pumps are the Aero Pump® AP3 dropper USD™ Art. nr. 3583/2007 pump head, and the Pfeiffer® SAP 78046 and SAP 75646 pump heads. The Aero Pump® AP3 dropper USD™ Art. nr. 3583/2007 pump head is shown in FIG. 3.

In one embodiment the pump 301 comprises a pump head 303 in which a nozzle 304 is arranged. The pump head 303 is also arranged with means 305 for a user to engage the pump head 303 and move it towards the container 302. In this embodiment the engaging means 305 is a flange 305 protruding from the head 301.

Inside the pump head 303 is a channel 307 through which the fluid to be dispensed can travel. The pump head 303 is movably coupled to a base or pump housing 306. In one embodiment the pump head 303 is biased away from the pump housing 306 by a spring 308. The spring 308 connects to a piston 309 that is arranged inside the pump house 306. As pressure is applied to the flange 305 in a direction towards the container 302 the pump head 303 compresses the spring 308. The spring 308 is compressed enough so that the force applied to the spring 308 is propagated through the spring 308 and acts on the piston 309 causing it to move.

The force required to move the piston 309 depends on a number of criteria such as for example, the spring coefficient of the spring 308 and the friction between the piston 309 and the pump housing 306 as well as the friction between the pump head 303 and the pump housing 306.

Piston 309 in turn is biased by a second spring 310. The force required to move piston 309 is therefore also dependant on the properties of the second spring 310. The piston 309 is sealably arranged in one end of a chamber 311 so that the piston 309 seals on end of the chamber 311. As the piston 309 moves inside the chamber 311 it reduces the volume of the chamber 311 thereby compressing any fluid contained in the chamber 311 causing it to travel up through a channel 307′ in the piston 309 and further through the head channel 307 and out through the nozzle 304.

The nozzle 304 is arranged to deliver a jet of the fluid and not a spray. The use of a jet advantageously enables the fluid to reach further through the nasal cavity and thus allows more of the fluid to reach the pharynx of the person the fluid is being administered to. The disadvantages of using a spray include that most of the fluid would only reach the nose 104 and nasal cavity 105 of the person the fluid is being administered to and the fluid would thus not reach its intended target area and have little or none of the wanted or desired effect.

A one-way valve 312, which in this embodiment is a ball valve 312, is arranged at another end of the chamber 311 in relation to the piston 309. In this embodiment the ball valve 312 is arranged at an opposite side from the piston 309. The ball valve 312 is arranged to allow flow into the chamber 311 but not out of the chamber 311. The chamber 311 is connected to a second chamber 313, which in turn is connected to the container 302 via a one-way valve 314. As the piston 309 is biased away from the chamber 311, the pressure on the flange 305 is released, and an under pressure in the chamber 311 builds up causing the ball-valve 312 to open and allow new fluid to flow into the chamber 311. The spring coefficient of spring 310 must therefore be high enough to allow the ball valve 312 to open.

As the ball valve 312 opens to allow fluid from chamber 313 to enter chamber 311, the under pressure of chamber 311 is shared between the two chambers 311, 313 which causes the ball valve 314 to open and allow fluid from the container 302 to be drawn up into chambers 311 and 313.

The arrangement with the two springs 308 and 310 has the advantage that to allow fluid to be dispensed through the nozzle a deliberate force is required as it needs to move the pump head 303 first enough to compress spring 8 and then to compress spring 310. Using only one spring, the pump action is initiated whenever force is applied to the flange 305, and the dispenser would easily dispense fluid against the user's will.

In one embodiment the second chamber 314 is connected to the container 302 via a channel 315, which reaches to the bottom of the container 302 in order to ensure that the fluid in the container 302 is drawn into the second chamber 314. This arrangement enables the pump 301 to deliver the fluid from the container 302 out through the nozzle 304 even when the pump 301 is tilted at an angle.

One fluid type solution that may be used with such a pump is Asonor®. The pump 301 and the container 302 together make up a dispensing unit for carrying and dispensing a liquid solution. Such a dispensing unit may be advantageously used for delivering a liquid solution for reducing snoring.

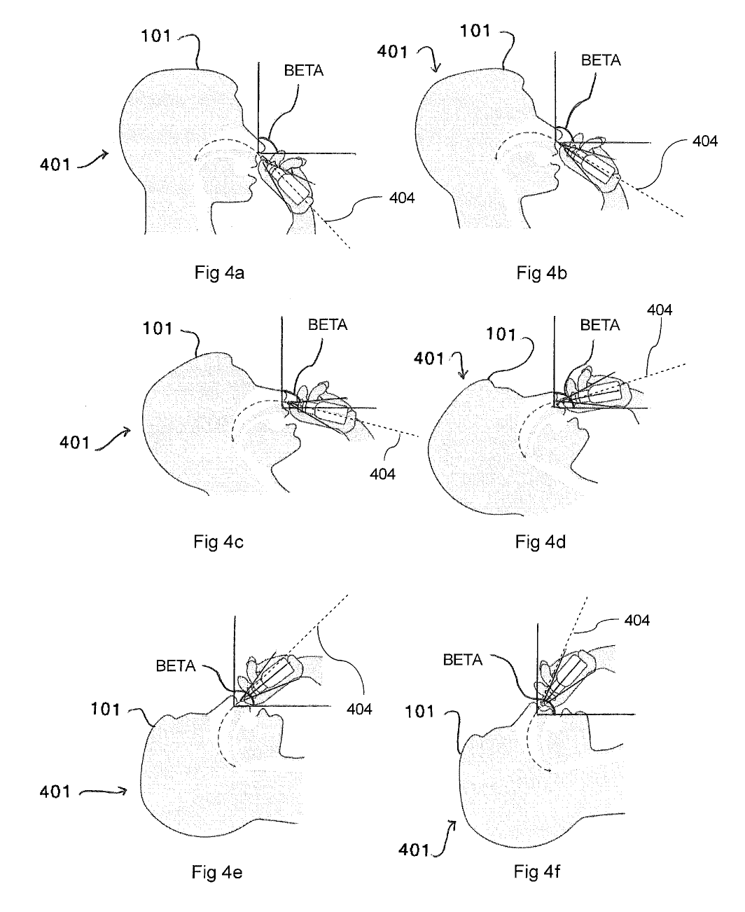

FIGS. 4a-4d show a series of positions taken by a person when dispensing the fluid. As shown in FIGS. 4a-4d, a second angle BETA indicates the angle of the user's nostrils relative the earth (or a horizontal plane). As a person lies down the second angle BETA increases. The angles BETA that gives the best results for providing the liquid to the pharynx 102 varies from person to person, but the second angle BETA is in the range or intervals of approximately −70 to 130 degrees; −60 to 120 degrees; −50 to 110 degrees; −40 to 100 degrees; −30 to 90 degrees; −20 to 80 degrees; −10 to 70 degrees; 0 to 60 degrees; 0 to 50 degrees; 0 to 40 degrees; 0 to 30 degrees; 20 to 60 degrees; 30 to 50 degrees and 40 to 45 degrees.

The first and second angles ALPHA and BETA are combined into a total angle at which the pump is being held relative the earth (or horizontal plane). Preferred values of the total angle are in the range of approximately 30 to 120 degrees.

In FIGS. 4a-4d, a line from the nostrils is indicated with a dashed line 404 and it is from this line 404 that the second angle BETA is measured relative to a horizontal plane.

FIG. 4a shows a person 401 sitting or standing upright—i.e., the head 101 is generally upright. As can be seen, the angle BETA is negative in this example.

FIG. 4b shows a person 401 leaning backwards slightly, i.e. the head 101 is angled backward. As can be seen the angle BETA is negative in this example.

FIG. 4c shows the head 101 of the person 401 leaning backwards. As can be seen, the angle BETA is negative and moving closer to being horizontal in this example.

FIG. 4d shows the head 101 of the person 401 leaning backwards heavily, or to a greater degree than shown in FIGS. 4a-4c. As can be seen the angle BETA is positive and closer to horizontal in this example.

FIG. 4e shows the head 101 of the person 401 substantially horizontal, such as for example, when the person 401 is lying down. As can be seen the angle BETA is positive in this example.

FIG. 4f shows an example where the head 101 of the person 401 is tilted back even further, such as when the person 401 is lying down and tilting his head further backwards. As can be seen, the angle BETA in this example is positive and close to vertical.

Thus, by realizing that the combination of tilting back ones head 101, and administering the fluid 106 from a pump 301 using a jet nozzle 304 one is able to advantageously reach the pharynx area 102 in a non-intrusive manner which is easy to use.

The manner is also highly useful and intuitive for reducing snoring as the position the person 401 must assume is similar to lying down which the person 401 would do in any case as he goes to sleep.

Different persons have different anatomy and shape and the angle of an individual tilting the head back giving the best result cannot be predicted. Some people will find it most comfortable and efficient to use the pump “upside down”, other people will find it most comfortable end efficient to use the pump “upside up” and some people will find it most comfortable end efficient to use the pump in both ways. This, together with the delivery and the jet stream is the reason for that this solution is the only one there will work satisfyingly to deliver a jet of liquid to the pharynx area in an easy to use and non-intrusive manner.

The method and the use of the pump is thus highly simple and intuitive to use and goes against existing prejudice in the field of how solutions can be administered.

Snoring can vary in intensity from being a mere annoying condition to a health threatening clinical condition.

The merely annoying condition is usually treated superficially and the clinical conditions are usually treated with therapeutic methods such as surgery as has been discussed above.

The superficial treatments are not as effective as the invasive therapeutic treatments and to avoid undergoing extensive therapeutic treatment such as surgery to alleviate the annoying snoring a non-therapeutic use of the pump as has been described may be applied. The use is non-therapeutic in the sense that it alleviates an annoying condition. The non-therapeutic use is not applied to a severe health-threatening condition.

Thus, by using the method and pump as described above a more efficient administration and treatment is possible using non-invasive means and is therefore more comfortable to use in situations where the more severe treatments are not needed.

The teaching of the present application has numerous advantages. Different embodiments or implementations may yield one or more of the following advantages. It should be noted that this is not an exhaustive list and there may be other advantages, which are not described herein. For example, one advantage of the teaching of this application is that the fluid may be dispensed without causing any unpleasantries. It is intuitive and easy to use. It does not require any expensive machinery. And it reaches an area that is difficult to reach without affecting other areas to any harmful or annoying extent.

Although the teaching of the present application has been described in detail for purpose of illustration, it is understood that such detail is solely for that purpose, and variations can be made therein by those skilled in the art without departing from the scope of the teaching of this application.

Features described in the preceding description may be used in combinations other than the combinations explicitly described.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

The term “comprising” as used in the claims does not exclude other elements or steps. The term “a” or “an” as used in the claims does not exclude a plurality. A unit or other means may fulfill the functions of several units or means recited in the claims.

What is claimed is:

1. A method of delivering a fluid to a pharynx area through a nasal cavity comprising:
bringing a pump to a nasal passage of a nose at an angle of approximately −90 to 130 degrees relative a horizontal ground plane; and
pumping a jet of the fluid through the nasal passage.

2. The method of claim 1 comprising tilting a head back and/or the person is lying down to allow access to the nose at the angle.

3. The method of claim 1 comprising delivering the fluid repeatedly through the pump while the head is being held at the angle.

4. The method of claim 1 wherein the fluid is a liquid solution for reducing swelling in the pharynx area or mucous membrane.

5. The method of claim 4 wherein the fluid is for reducing obstructions in an airway to reduce snoring.

6. The method of claim 1 wherein the angle is in at least one of the ranges of approximately −70 to 130; −60 to 120; −50 to 110; −40 to 100; −30 to 90; −20 to 80; −10 to 70; 0 to 60; 0 to 50; 0 to 40; 0 to 30; 20 to 60; 30 to 50 and 40 to 45 degrees.

7. A use of a pump to administer a liquid solution to a pharynx area through a nose and a nasal cavity by providing a jet of a liquid solution from the pump, wherein the pump is configured to provide a jet stream of the liquid solution into the nasal cavity while the pump is positioned at an angle of approximately −10 to 90 degrees relative a substantially horizontal ground plane.

8. The use according to claim 7, wherein the pump is configured to repeatedly provide the jet stream of liquid while being positioned at the angle.

9. The use according to claim 7, wherein the person is lying down or the head is tilted or leaned backwards to allow access to the nose at the angle.

10. The use according to claim 7, wherein the angle is in at least one of the ranges of approximately −70 to 130; −60 to 120; −50 to 110; −40 to 100; −30 to 90; −20 to 80; −10 to 70; 0 to 60; 0 to 50; 0 to 40; 0 to 30; 20 to 60; 30 to 50 and 40 to 45 degrees.

11. The use according to claim 7, wherein the use is a non-therapeutic treatment to reducing snoring and wherein the liquid solution is to reducing swelling in the pharynx area or mucous membrane to reduce obstructions in an airway of a person to reduce snoring.

12. A nasal fluid delivery system comprising:
a pump for delivering a liquid solution through a nose and nasal cavity to a pharynx area, the pump comprising:
a nozzle configured to generate a jet stream of the liquid solution, the pump being configured to deliver the jet stream of the liquid solution while the pump is positioned at an angle relative to a substantially horizontal ground plane.

13. The nasal fluid delivery system according to claim 12, wherein the liquid solution reduces swelling in the pharynx area or the mucous membrane to reduce snoring.

14. The nasal fluid delivery system according to claim 12, wherein the pump delivers the jet stream of liquid repeatedly while being held at the angle.

15. The nasal fluid delivery system according to claim 12 comprising:
a pump head movably connected to a pump housing and biased away from the pump housing by a first spring;
the spring being connected to a piston which is movably arranged in the pump housing;
the pump housing being connected to a first chamber arranged to house a portion of the liquid solution;
the piston being biased away from the chamber by a second spring and wherein the piston is configured to seal a first end of the chamber;
wherein the chamber is connected to a second chamber via a one-way valve, the second chamber being arranged to house the portion of the liquid solution and the one-way valve being configured to allow flow of the portion of the liquid solution into the first chamber; and
wherein the second chamber is connected to a container for carrying the liquid solution via a second one-way valve that is configured to allow a flow of the portion of the liquid solution into the second chamber.

16. A nasal fluid delivery system according to claim 12 comprising a dispenser unit comprising the pump and a container carrying the liquid solution, wherein the liquid solution is for reducing snoring.

17. The nasal fluid delivery system of claim 16 wherein the angle is substantially vertical relative to a substantially horizontal ground plane.

18. The nasal fluid delivery system of claim 17 wherein the nozzle is pointed substantially downwards.

19. The nasal fluid delivery system according to claim 18 wherein the pump is configured to deliver multiple doses of the liquid solution while the nozzle is pointed substantially downwards.

20. The nasal fluid delivery system of claim 19 wherein the liquid solution is an anti-snoring solution.
